(19) United States
(12) Patent Application Publication
(10) Pub. No.: US 2004/0197355 A1
(43) Pub. Date: Oct. 7, 2004

(54) Cosmetic composition comprising calcuim carbonate particles and a combination of surfactants

(76) Inventors: Beatrice Perron, Jouy en Josas (FR); Serge Restle, Saint-Prix (FR); Franck Giroud, Clichy (FR); Henri Samain, Bievres (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/479,175

(22) PCT Filed: May 31, 2002
(86) PCT No.: PCT/FR02/01845

(30) Foreign Application Priority Data
Dec. 1, 2003 (FR) 01/01845

(51) Int. Cl. A61K 7/00

(52) U.S. Cl. 424/401

(57) ABSTRACT

The invention concerns a cosmetic composition, in particular for hair care, comprising, in a cosmetically acceptable medium: (a) solid particles containing at least 10 wt. % of carbonate calcium; (b) a combination of at least two surfactants, selected among combinations: of at least an anionic surfactant (i) and of at least an amphoteric surfactant (ii) or of at least an anionic surfactant (i) and of at least a nonionic surfactant (iii); (c) a cationic polymer whereof the cationic charge density is not more than 7 meq/g and, preferably, not less than 0.05 meq/g.

The present invention relates to a cosmetic composition, especially the hair composition, containing, in a cosmetically acceptable medium, calcium carbonate particles and a combination of surfactants. The invention is also directed toward a cosmetic hair treatment process comprising the application of this composition, and to its use as a rinse-out hair product.

It is common practice to use detergent hair compositions (or shampoos) based essentially on standard surfactants especially of anionic, nonionic and/or amphoteric type, but more particularly of anionic type, for cleansing and/or washing the hair. These compositions are applied to wet hair and the lather generated by massaging or rubbing with the hands makes it possible, after rinsing with water, to remove the various types of soiling initially present on the hair.

These base compositions do indeed have good washing power, but the intrinsic cosmetic properties associated therewith remain quite poor, especially due to the fact that the relatively aggressive nature of such a cleansing treatment can in the long run result in more or less pronounced damage to the hair fibers, which is associated in particular with the gradual removal of the lipids or proteins contained in them or at their surface.

Thus, to improve the cosmetic properties of the above compositions, and more particularly of those intended to be used on sensitized hair (i.e. hair that is damaged or embrittled especially due to the chemical action of atmospheric agents and/or hair treatments such as permanent-waving, dyeing or bleaching), it is now common practice to introduce into these compositions additional cosmetic agents known as conditioners, which are intended mainly to repair or limit the harmful or adverse effects induced by the various treatments or attacking factors to which the hair fibers are more or less repeatedly subjected. These conditioners may, of course, also improve the cosmetic behavior of natural hair.

The conditioners most commonly used to date in shampoos are cationic polymers, silicones and/or silicone derivatives, which in fact give washed, dry or wet hair markedly increased ease of disentangling and softness when compared with that which may be obtained with the corresponding cleansing compositions from which they are absent.

However, these cosmetic advantages are unfortunately also accompanied, on the dried hair, by certain cosmetic effects that are considered undesirable, i.e. lankness of the hairstyle and a lack of smoothing.

In addition, the use of cationic polymers for this purpose presents various drawbacks. On account of their high affinity for the hair, some of these polymers become deposited in large amount during repeated use, and lead to adverse effects such as an unpleasant, laden feel, stiffening of the hair, and adhesion between the fibers, which affects the styling. These drawbacks are accentuated in the case of fine hair, which lacks liveliness and volume.

It has already been proposed to use particles in rinse-out compositions, so as to improve the feel and appearance of the hair. By weight of illustration, U.S. Pat. No. 5,334,376 proposes the addition of calcium carbonate particles to hair conditioning compositions containing a silicone, a fatty alcohol and an amide.

It has also been proposed, in patent application DE 199 46 784, to use particles of the various oxides, hydroxides, carbonates, silicates or phosphates in hair compositions, to reduce the greasy appearance of the hair. It is generally envisioned to combine these particles with standard shampoo ingredients.

However, despite the progress recently made in the field of rinse-out hair products and especially shampoos, these products are not truly totally satisfactory, and as such there is currently still a strong need for novel products that show better performance qualities as regards one or more of the cosmetic properties.

The Applicant has discovered, surprisingly and unexpectedly, that by carefully selecting the surfactant base and the conditioner, combined with calcium carbonate particles, it is possible to improve the results obtained with cosmetic products, especially rinse-out hair products, in terms of cosmetic properties and shaping properties. In particular, the hair is given texture (increased sensation of thickness) and better hairstyle hold.

One subject of the invention is a cosmetic composition, especially a hair composition, comprising, in a cosmetically acceptable medium:

(a) solid particles containing at least 10% by weight of calcium carbonate;

(b) a combination of at least two surfactants, chosen from combinations:

of at least one anionic surfactant (i) and of at least one amphoteric surfactant (ii), or

of at least one anionic surfactant (i) and of at least one nonionic surfactant (iii);

(c) a cationic polymer whose cationic charge density is less than or equal to 7 meq/g and preferably greater than or equal to 0.05 meq/g.

Preferably, the concentration weight ratio of the anionic surfactant(s) to the nonionic and/or amphoteric surfactants is greater than 1.

Another subject of the present invention consists of a cosmetic hair treatment process using the composition according to the invention.

A subject of the invention is also the use of the cosmetic hair composition especially in rinse-out hair application.

Other subjects, characteristics, aspects and advantages of the invention will emerge even more clearly on reading the description and the various examples that follow.

Calcium Carbonate Particles

The particles containing at least 10% by weight of calcium carbonate preferably have a number-average primary size of between 2 nm and 2 μm, more preferably between 5 and 500 nm and even more preferably between 10 and 250 nm.

The particles according to the invention may be, for example, in any form, for example in the form of spheres, flakes, needles, platelets or totally random forms.

In accordance with the present invention, the particle may be a solid particle formed entirely from calcium carbonate. Calcium carbonate may also totally or partially constitute the core of the particle, this core being coated with another constituent, for instance an oxide, a silicate or a metal. Calcium carbonate may also exclusively form the coating of a substrate of different chemical constitution, for instance an oxide, a silicate or a metal.

For the purposes of the present invention, the expression “primary particle size” means the maximum size that it is possible to measure between two diametrically opposite points on an individual particle. The size may be determined, for example, by transmission electron microscopy or by measuring the specific surface area by the BET method or alternatively using a laser granulometer.

When the particles are formed by calcium carbonate and other fillers, the calcium carbonate is in free form and does not form chemical bonds with the other fillers. It is then a matter of an alloy between the calcium carbonate and other fillers, especially with metal or metalloid oxides, obtained in particular by thermal fusion of these various constituents.

When the particles containing at least 10% by weight of calcium carbonate also comprise a metal or metalloid oxide, this oxide is chosen especially from silicon oxide, boron oxide and aluminum oxide.

Preferably, the particles contain at least 50% by weight of calcium carbonate and better still at least 70% by weight, and particles consisting of more than 90% by weight of calcium carbonate are particularly preferred according to the present invention.

Even more advantageously, the particles containing at least 10% by weight of calcium carbonate are particles of substantially pure calcium carbonate.

The particles containing calcium carbonate according to the invention are used especially in an amount of between 0.01% and 30% by weight and preferably between 0.05% and 5% by weight relative to the total weight of the composition.

The calcium carbonate that is suitable for use in the compositions of the present invention may be of natural origin or may be of synthetic origin. In the latter case, it may be obtained from calcium oxide, calcium peroxide, calcium acetate or calcium ethoxide.

The composition according to the invention may also contain other types of particles, for example titanium oxide, zinc oxide or aluminum oxide particles.

The surfactants that are suitable for use in the present invention are especially the following:

(i) Anionic Surfactant(s):

As examples of anionic surfactants which can be used, alone or as mixtures, in the context of the present invention, mention may be made in particular of salts (in particular alkaline salts, especially sodium salts, ammonium salts, amine salts, amino alcohol salts or magnesium salts) of the following compounds: alkyl sulfates, alkyl ether sulfates, alkylamido ether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates and N-acyltaurates, the alkyl or acyl radical of all of these various compounds preferably containing from 12 to 20 carbon atoms, and the aryl radical preferably denoting a phenyl or benzyl group. Among the anionic surfactants which can also be used, mention may also be made of fatty acid salts such as the salts of oleic, ricinoleic, palmitic and stearic acids, coconut oil acid or hydrogenated coconut oil acid; acyl lactylates in which the acyl radical contains 8 to 20 carbon atoms. Weakly anionic surfactants can also be used, such as alkyl-D-galactosiduronic acids and their salts, as well as polyoxyalkylenated ether carboxylic acids and their salts, in particular those containing from 2 to 50 ethylene oxide groups, and mixtures thereof. The anionic surfactants of the polyoxyalkylenated carboxylic ether acid or salt type are, in particular, those that correspond to formula (I) below:

R₁—(—OC₂H₄—)ₙ—OCH₂COOA (1)

in which:

R₁ denotes an alkyl or alkylaryl group and n is an integer or decimal (average value) which can range from 2 to 24 and preferably from 3 to 10, the alkyl radical having between 6 and 20 carbon atoms approximately, and aryl preferably denoting phenyl,

A denotes H, ammonium, Na, K, Li or Mg or a monoethanolamine or triethanolamine residue. Mixtures of compounds of formula (1), in particular mixtures in which the groups R₁ are different, can also be used.

Among the anionic surfactants, it is preferred according to the invention to use more particularly alkyl sulfate salts and alkyl ether sulfate salts and mixtures thereof.

(ii) Amphoteric Surfactant(s):

The amphoteric surfactants may especially be aliphatic secondary or tertiary amine derivatives in which the aliphatic radical is a linear or branched chain containing 8 to 22 carbon atoms and containing at least one water-soluble anionic group (for example carboxylate, sulfonate, sulfate, phosphate or phosphonate); mention may also be made of (C₈-C₂₀)alkylbetaines, sulfobetaines, (C₈-C₂₀)alkylamido(C₁-C₆)alkylbetaines or (C₈-C₂₀)alkylamido(C₁-C₆)alkylsulfobetaines.

Among the amine derivatives, mention may be made of the products sold under the name Miranol, as described in U.S. Pat. Nos. 2,528,378 and 2,781,354, and having the structures:

R₂—CONHCH₂CH₂—N(R₃)(R₄)(CH₂COO—) (2)

in which: R₂ denotes an alkyl radical derived from an acid R₂—COOH present in hydrolysed coconut oil, a heptyl, nonyl or undecyl radical, R₃ denotes a β-hydroxyethyl group and R₄ denotes a carboxymethyl group; and

R₅—CONHCH₂CH₂—N(B)(C) (3)

in which:

B represents —CH₂CH₂OX′, C represents —(CH₂)_{z}—Y′, with z=1 or 2,

X′ denotes the —CH₂CH₂—COOH group or a hydrogen atom,

Y′ denotes —COOH or the —CH₂—CHOH—SO₃H radical,

R₅ denotes an alkyl radical of an acid R₉—COOH present in coconut oil or in hydrolysed linseed oil, an alkyl radical, in particular a C₇, C₉, C₁₁ or C₁₃ alkyl radical, a C₁₇ alkyl radical and its iso form, an unsaturated C₁₇ radical.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

By way of example, mention may be made of the cocoamphodiacetate sold under the trade name Miranol C2M concentrate by the company Rhône-Poulenc.

(iii) Nonionic Surfactant(s):

The nonionic surfactants are, themselves also, compounds that are well known per se (see in particular in this respect “Handbook of Surfactants” by M. R. Porter, published by Blackie & Son (Glasgow and London), 1991, pp. 116-178). They can be chosen in particular from (non-limiting list) polyethoxylated, polypropoxylated or polyglycerolated fatty acids, alkylphenols, α-diols or alcohols having a fatty chain containing, for example, 8 to 18 carbon atoms, it being possible for the number of ethylene oxide or propylene oxide groups to range in particular from 2 to 50 and for the number of glycerol groups to range in particular from 2 to 30. Mention may also be made of copolymers of ethylene oxide and of propylene oxide, condensates of ethylene oxide and of propylene oxide with fatty alcohols; polyethoxylated fatty amides preferably having from 2 to 30 mol of ethylene oxide, polyglycerolated fatty amides containing on average 1 to 5, and in particular 1.5 to 4, glycerol groups; polyethoxylated fatty amines containing preferably 2 to 30 mol of ethylene oxide; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides. It will be noted that the alkylpolyglycosides constitute nonionic surfactants that are particularly suitable in the context of the present invention.

Advantageously, the relative concentrations of the surfactants in the composition are such that:

the anionic surfactants (i) are present in a proportion of from 2% to 50% by weight and preferably from 3% to 20% by weight relative to the total weight of the composition;

the amphoteric surfactants (ii) are present in a proportion of from 1% to 50% by weight and preferably from 1% to 20% by weight relative to the total weight of the composition;

the nonionic surfactants (iii) are present in a proportion of from 1% to 50% by weight and preferably from 1% to 20% by weight relative to the total weight of the composition.

The anionic surfactant that is preferably used is chosen from sodium, triethanolamine or ammonium (C₁₂-C₁₄)alkyl sulfates, sodium, triethanolamine or ammonium (C₁₂-C₁₄)alkyl ether sulfates oxyethylenated with 2.2 mol of ethylene oxide, sodium, triethanolamine or ammonium (C₁₂-C₁₄)alkylamido sulfates, sodium cocoyl isethionate and sodium α-(C₁₄-C₁₆)olefin sulfonate, and mixtures thereof, with:

either an amphoteric surfactant such as the amine derivatives known as disodium cocoamphodipropionate or sodium cocoamphopropionate sold especially by the company Rhône-Poulenc under the trade name “Miranol C2M CONC” as an aqueous solution containing 38% active material, or under the name Miranol C32;

or an amphoteric surfactant of zwitterionic type such as alkylamidobetaines and alkylbetaines, in particular the cocobetaine sold under the name “Dehyton AB 30” as an aqueous solution containing 32% AM by the company Henkel.

Cationic Polymer

The cationic polymers used in accordance with the invention generally have a weight-average molecular weight of at least 5000, preferably of at least 10 000, and less than 10 000 000, and more particularly ranging from 100 000 to 2 000 000. They generally have units containing a nitrogen atom, such as quaternary ammonium or amino units or mixtures thereof. Their cationic charge density is less than or equal to 7 meq/g and preferably greater than or equal to 0.05 meq/g and more preferably between 0.5 and 7 meq/g. The charge density can be determined according to the Kjeldahl method. It generally corresponds to a pH of about 3 to 9.

Among the cationic polymers which can be used according to the invention, mention may be made of copolymers of vinyl monomers having amine or quaternary ammonium functions with water-soluble monomers containing ethylenic unsaturation, such as acrylamide, methacrylamide, alkyl- or dialkyl(meth)acrylamides, alkyl(meth)acrylates, vinylcaprolactone, vinylpyrrolidone; or alternatively other monomers such as vinyl esters, vinyl alcohol, maleic anhydride, propylene glycol, ethylene glycol. The alkyl or dialkyl groups of the amine or ammonium functions are preferably C₁-C₉ and more preferably C₁-C₃.

The amines can be primary, secondary or tertiary. Secondary and tertiary amines are preferred.

The amino-substituted vinyl monomers can be polymerized in their amine form and then optionally quaternized. The amines can also be quaternized after formation of the polymer. For example, the tertiary amine functions can be quaternized by reaction with a salt of formula R′X in which R′ is a short-chain (preferably C₁-C₇ and more particularly C₁-C₃) alkyl radical and X is an anion forming a water-soluble salt with the quaternary ammonium.

Among the vinyl monomers containing amine or quaternary ammonium functions, mention may be made, for example, of vinyl compounds substituted with a group such as dialkylaminoalkyl(meth)acrylate, monoalkyl-aminoalkyl(meth)acrylate; trialkylmethacryloxyalkyl-ammonium salts; diallylic salts of quaternary ammonium; quaternary vinyl monomers having rings bearing nitrogen atoms, such as pyridinium, imidazolium, quaternized pyrrolidone, for instance alkylvinylimidazolium, alkylvinylpyridinium, quaternary alkylvinylpyrrolidone salts. The alkyl portions of these monomers are preferably C₁-C₃ alkyls and more preferably C₁ or C₂ alkyls.

Mention may also be made, as amino-substituted vinyl monomers, of dialkylaminoalkyl (meth)acrylates and dialkylaminoalkyl(meth)acrylamides. The alkyl or dialkyl groups are preferably C₁-C₉ and more preferably C₁-C₃.

The cationic polymers of the invention can comprise mixtures of vinyl monomers derived from amines and/or of vinyl monomers derived from quaternary ammoniums and/or of other compatible monomers. Mention may be made, by way of example, of:

copolymers of 1-vinylpyrrolidone and of a salt of 1-vinyl-3-methylimidazolium (for example the chloride) (known as Polyquaternium-16 in the CTFA dictionary), such as those sold under the name Luviquat by the company BASF;

copolymers of 1-vinyl-2-pyrrolidone and of dimethylaminoethyl methacrylate (known as Polyquaternium-11 in the CTFA dictionary), such as those sold under the name Gafquat (for example Gafquat 755N) by the company GAF Corporation;

dimethyldiallylammonium chloride homopolymers (Polyquaternium-5 in the CTFA dictionary) and copolymers of acrylamide and of dimethyldiallyl-ammonium chloride (Polyquaternium-7 in the CTFA dictionary), such as those sold under the name Merquat 550 and Merquat S by the company Merck;

mineral acid salts of aminoalkyl esters of homo- and copolymers of unsaturated carboxylic acids having from 3 to 5 carbon atoms, such as those described in U.S. Pat. No. 4,009,256.

Among the cationic polymers that can be used, mention may also be made of cationic polysaccharides such as cationic cellulose derivatives and cationic starch derivatives.

Among the cationic polysaccharides, mention may be made of polymers of formula:

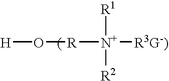

in which:

H is an anhydroglucose residue such as starch or a cellulosic anhydroglucose residue;

R is an alkylene, an oxyalkylene, a polyoxyalkylene or a hydroxyalkylene or mixtures thereof;

R¹, R² and R³, which may be identical or different, denote an alkyl, aryl, alkylaryl, arylalkyl, alkoxyalkyl or alkoxyaryl group; each group containing up to 18 carbon atoms and the total number of carbon atoms per cationic unit is preferably less than or equal to 20;

G⁻ is an anion resulting from the quaternization of the amine NR¹R²R³.

Among the cationic cellulose polymers, mention may be made of those sold by the company Amerchol Corp. under the names JR and LR, such as the quaternary hydroxyethylcellulose salts obtained by reaction with an epoxide substituted with a trimethylammonium (Polyquaternium-10 in the CTFA dictionary). Mention may also be made of the quaternary hydroxyethylcellulose salts obtained by reaction with an epoxide substituted with lauryldimethylammonium (Polyquaternium-24 in the CTFA dictionary), such as those sold under the name Polymer LM200 by Amerchol Corp.

As cationic polymers which can be used according to the invention, mention may also be made of cationic guar gum derivatives, such as hydroxypropyltrimonium guar chloride sold under the name Jaguar by the company Celanese Corp.

Mention may also be made of quaternary cellulose ethers, such as those described in U.S. Pat. No. 3,962,418 and etherified copolymers of cellulose and of starch, such as those described in U.S. Pat. No. 3,958,581.

The cationic polymers of the invention are present in the compositions in proportions preferably ranging from 0.01 to 5% by weight and preferably from 0.1 to 3% by weight relative to the total weight of the composition.

Composition

The cosmetically acceptable aqueous medium may consist solely of water or of a mixture of water and one or more cosmetically acceptable solvents, or of one or more cosmetically acceptable solvents, such as a C₁-C₄ lower alcohol, for instance ethanol, isopropanol, tert-butanol or n-butanol; alkylene glycols, for instance propylene glycol, and glycol ethers.

The cosmetically acceptable medium preferably contains water.

The detergent compositions according to the invention have a final pH generally of between 3 and 10. Preferably, this pH is between 4 and 8. The pH can be adjusted to the desired value conventionally by adding a base (organic or inorganic) into the composition, for example aqueous ammonia or a primary, secondary or tertiary(poly)amine such as monoethanolamine, diethanolamine, triethanolamine, isopropanolamine or 1,3-propanediamine, or alternatively by adding an acid, preferably a carboxylic acid such as, for example, citric acid.

The compositions in accordance with the invention can contain, in addition to the combination defined above, viscosity modifiers such as electrolytes, or thickeners (associative or non-associative). Mention may be made in particular of sodium chloride, sodium xylenesulfonate, scleroglucans, xanthan gums, fatty acid alkanolamides, alkanolamides of carboxylic acid alkyl ether optionally oxyethylenated with up to 5 mol of ethylene oxide, such as the product sold under the name “Aminol A15” by the company Chem Y, crosslinked polyacrylic acids and crosslinked acrylic acid/C₁₀-C₃₀ alkyl acrylate copolymers. These viscosity modifiers are used in the compositions according to the invention in proportions that may range up to 10% by weight relative to the total weight of the composition.

The compositions in accordance with the invention can also contain up to 5% of pearlescent agents or opacifiers that are well known in the state of the art, such as, for example, fatty alcohols greater than C16, acyl derivatives containing a fatty chain, such as monostearates or distearates of ethylene glycol or of polyethylene glycol, fatty-chain ethers such as distearyl ether or 1-(hexadecyloxy)-2-octadecanol.

The compositions in accordance with the invention may also optionally comprise at least one additive chosen from foam synergists such as C₁₀-C₁₈ 1,2-alkanediols or fatty alkanolamides derived from monoethanolamine or from diethanolamine, silicone or nonsilicone sunscreens, cationic surfactants, anionic, nonionic, amphoteric or cationic polymers other than those of the invention, proteins, protein hydrolysates, hydroxy acids, vitamins, provitamins such as panthenol, and volatile or nonvolatile, linear or cyclic, crosslinked or noncrosslinked, organomodified or non-organomodified silicones.

Needless to say, a person skilled in the art will take care to select this or these optional additional compound(s) and/or the amounts thereof such that the advantageous properties intrinsically associated with the combination in accordance with the invention are not, or are not substantially, adversely affected by the envisioned addition(s).

These additives are optionally present in the composition according to the invention in proportions that may range from 0.0001% to 20% by weight relative to the total weight of the composition. The amount of each additive is readily determined by a person skilled in the art depending on its nature and its function.

These compositions may be in the form of more or less thickened liquids, creams or gels, and they are mainly suitable for washing and caring for keratin materials, in particular the hair and the skin and even more particularly the hair.

When the compositions in accordance with the invention are used as standard shampoos, they are simply applied to wet hair and the lather generated by massaging or friction with the hands is then removed, after an optional leave-in time, by rinsing with water, the operation possibly being repeated one or more times.

A further subject of the invention is a process for washing and conditioning keratin materials such as in particular the hair, which comprises applying a composition as defined hereinabove, then rinsing with water after an optional leave-in time.

The compositions according to the invention are preferably used as shampoos for washing and conditioning the hair, and in this case they are applied to wet hair in amounts that are effective to wash it, this application being followed by rinsing with water.

The compositions in accordance with the invention may also be used as shower gels for washing and conditioning the hair and/or the skin, in which case they are applied to the wet skin and/or hair and are rinsed off after application.

The compositions of the invention may also be used in leave-in mode, and in particular in lotions, gels, mousses or aerosols.

Concrete, but in no way limiting, examples illustrating the invention will now be given.

### EXAMPLES

The two shampoos below are prepared:

| | |
|---|---|
| Shampoo A | |
| Triethanolamine lauryl sulfate at a concentration | 31.3% |
| of 40% AM | |
| Cocoylamidopropylbetaine (1) at a concentration | 6.6% |
| of 38% AM | |
| Cationic polymer (2) | 3.1% |
| Calcium carbonate powder (3) | 3% |
| qs pH = 7 | |
| qs water = 100 | |
| Shampoo B | |
| Triethanolamine lauryl sulfate at a concentration | 31.3% |
| of 40% AM | |
| (C1/C9)Alkyl polyglucoside [1,4] (4) | 6.3% |
| Cationic polymer (2) | 3.1% |
| Calcium carbonate powder (3) | 3% |
| qs pH = 7 | |
| qs water = 100 | |

(1) as an aqueous solution
(2) 8% protected 50/50 dimethyldiallylammonium chloride/acrylamide as an aqueous solution
(3) Omya Pur 3S sold by Omya
(4) as an aqueous 40% solution

The composition has a pleasant texture when applied to wet hair. It is easily rinsed out. The wet hair is not laden and is easy to shape.

1-18. (canceled)

19. A cosmetic composition, comprising, in a cosmetically acceptable medium:
(a) solid particles comprising at least 10% by weight of calcium carbonate;
(b) a combination of at least two surfactants, selected from the group consisting of at least one anionic surfactant (i), at least one amphoteric surfactant (ii), of at least one nonionic surfactant (iii), and mixtures thereof; and
(c) a cationic polymer whose cationic charge density is of from 0.05 meq/g to 7 meq/g.

20. The composition as claimed in claim 19, wherein the solid particles comprising at least 10% by weight of calcium carbonate have a number-average primary size of between 2 nm and 2 μm.

21. The composition as claimed in claim 19, wherein the solid particles comprising at least 50% by weight of calcium carbonate.

22. The composition as claimed in claim 19, wherein the solid particles are particles of substantially pure calcium carbonate.

23. The composition as claimed in claim 19, wherein the solid particles containing calcium carbonate are used in an amount of between 0.01% and 30% by weight relative to the total weight of the composition.

24. The composition as claimed in claim 19, comprising at least one anionic surfactant (i); wherein at least one anionic surfactant (i) is present in a proportion of from 2% to 50% by weight relative to the total weight of the composition.

25. The composition as claimed in claim 19, comprising at least one amphoteric surfactant (ii); wherein at least one amphoteric surfactant (ii) is present in a proportion of from 1% to 50% by weight relative to the total weight of the composition.

26. The composition as claimed in claim 19, comprising at least one nonionic surfactant (iii); wherein at least one nonionic surfactant (iii) is present in a proportion of from 1% to 50% by weight relative to the total weight of the composition.

27. The composition as claimed in claim 19, comprising at least one anionic surfactant (i) and at least one nonionic surfactant (ii) and/or at least one amphoteric surfactant (iii); wherein the concentration weight ratio of the total anionic surfactant (i) to the total nonionic surfactant (ii) and/or total amphoteric surfactant (iii) is greater than 1.

28. The composition as claimed in claim 19, comprising at least one anionic surfactant (i); wherein at least one anionic surfactant (i) is selected from the group consisting of a M(C₁₂-C₁₄)alkyl sulfate, a M (C₁₂-C₁₄)alkyl ether sulfate oxyethylenated with 2.2 mol of ethylene oxide, a M(C₁₂-C₁₄)alkylamido sulfate, a sodium cocoyl isethionate, and a sodium α-(C₁₄-C₁₆)olefin sulfonate; wherein M is selected from the group consisting of sodium, triethanolammonium, and ammonium.

29. The composition as claimed in claim 19, comprising amphoteric surfactant (ii); wherein at least one amphoteric surfactant (ii) is selected from the group consisting of an aliphatic secondary amine derivative; wherein the aliphatic radical is a linear or branched chain comprising 8 to 22 carbon atoms and comprising at least one anionic group, a tertiary amine derivative; wherein the aliphatic radical is a linear or branched chain comprising 8 to 22 carbon atoms and comprising at least one anionic group, a (C₈-C₂₀)alkylbetaine, a sulfobetaine, a (C₈-C₂₀)alkylamido(C₁-C₆)alkylbetaine, and a (C₈-C₂₀)alkylamido(C₁-C₆)alkylsulfobetaine.

30. The composition as claimed in claim 19, comprising at least one nonionic surfactant (iii); wherein at least one nonionic surfactant (iii) is selected from the group consisting of a polyethoxylated fatty acid; wherein the number of ethylene oxide groups range from 2 to 50, a polypropoxylated fatty acid; wherein the number of propylene oxide groups range from 2 to 50, a polyglycerolated fatty acid; wherein the number of glycerol groups range from 2 to 30, an alkylphenol, an α-diol, and an alcohol having a fatty chain comprising 8 to 18 carbon atoms.

31. The composition as claimed in claim 19, wherein the cationic charge density of the cationic polymer (c) ranges from 0.5 to 7 meq/g.

32. The composition as claimed in claim 19, wherein the cationic polymer (c) is selected from the group consisting of a) a copolymer of vinyl monomers containing primary, secondary or tertiary amine functions or quaternary ammonium functions; b) a cationic polysaccharide derived from starch or derived from cellulose; c) a cationic polymer derived from guar gum; and d) a quaternary cellulose ether, and copolymers of etherified cellulose and of starch.

33. The composition as claimed in claim 19, wherein the cationic polymer (c) is present in proportions ranging from 0.01% to 5% by weight relative to the total weight of the composition.

34. The composition as claimed claim 19, which further comprises at least one additive selected from the group consisting of a foam synergist, which is selected from the group consisting of a C₁₀-C₁₈ 1,2-alkanediol; a fatty alkanolamide; a silicone sunscreen; a nonsilicone sunscreen; a cationic surfactant; an anionic polymer unlike (i); a nonionic polymer unlike (ii); a cationic polymer unlike (iii); a protein; a protein hydrolysate; a hydroxy acid; a vitamin; a provitamin; a volatile silicone; a nonvolatile silicone; a linear silicone; a cyclic silicone; a crosslinked silicone; a noncrosslinked silicone; an organomodified silicone; a non-organomodified silicone; and mixtures thereof.

35. A shampoo, which comprises:
the composition as claimed in claim 19.

36. A process for washing and conditioning the hair, which comprises:
applying an effective amount of a composition as defined in claim 19, and then
rinsing with water after an optional time period.
